(19) United States
(12) Patent Application Publication
(10) Pub. No.: US 2023/0348909 A1
(43) Pub. Date: Nov. 2, 2023

(54) DYSTROPHIN EXON SKIPPING OLIGONUCLEOTIDES

(71) Applicant: BioMarin Pharmaceutical Inc., Novato, CA (US)

(72) Inventors: Steven Michael Froelich, Tiburon, CA (US); Josh Christopher Woloszynek, Novato, CA (US); Jenna-Marie Magat, Pacifica, CA (US); Judith Christina Theodora van Deutekom, Dordrecht (NL); Peter Christian de Visser, Leiden (NL); Nicole Anne Datson, Oegstgeest (NL)

(21) Appl. No.: 18/192,480

(22) Filed: Mar. 29, 2023

Related U.S. Application Data
(60) Provisional application No. 63/362,189, filed on Mar. 30, 2022.

(51) Int. Cl. C12N 15/113; A61P 21/00

(52) U.S. Cl. CPC C12N 15/113; A61P 21/00; C12N 2310/321; C12N 2310/3231; C12N 2310/3341; C12N 2310/315; C12N 2310/11

(57) ABSTRACT

Provided herein are antisense oligonucleotides (AONs) that induce skipping of exon 51 of human dystrophin pre-mRNA, and pharmaceutically acceptable derivatives thereof. Also provided are pharmaceutical compositions containing the AONs and methods of using the AONs and compositions for treating a subject with Duchenne muscular dystrophy.

### RELATED APPLICATION

This application claims the benefit of priority of U.S. Provisional Patent Application No. 63/362,189, filed Mar. 30, 2022. The disclosure of the above application is incorporated by reference herein in its entirety.

### SEQUENCE LISTING

This application contains a Sequence Listing, which is being submitted herewith as an XML filed named “035104US_SL.xml”, created on Mar. 23, 2023, size 85,187 bytes, which is incorporated by reference herein in its entirety.

### FIELD

Provided herein are antisense oligonucleotides for use in compositions and methods of dystrophin exon skipping and treating Duchenne muscular dystrophy.

### BACKGROUND

Antisense oligonucleotides (AONs) are in (pre)clinical development for many diseases and conditions, including cancer, inflammatory conditions, cardiovascular disease and neurodegenerative and neuromuscular disorders. Their mechanism of action is aimed at various targets, such as RNaseH-mediated degradation of target RNA in the nucleus or cytoplasm, at splice-modulation (exon inclusion or skipping) in the nucleus, or at translation inhibition by steric hindrance of ribosomal subunit binding in the cytoplasm. Splice-modulating or splice-switching AONs were first described for correction of aberrant splicing in human β-globin pre-mRNAs (Dominski and Kole PNAS, 1993, 90(18):8673-8677), and are currently being studied for a variety of genetic disorders.

AONs have been extensively studied in the treatment of the neuromuscular disorders Duchenne muscular dystrophy (DMD) and Becker muscular dytrophy (BMD). Duchenne muscular dystrophy (DMD) and Becker muscular dystrophy (BMD) are the most common childhood forms of muscular dystrophy. DMD is a severe, lethal neuromuscular disorder resulting in a dependency on wheelchair support before the age of 12 and patients often die before the age of thirty due to respiratory or heart failure. It is caused by reading frame-shifting deletions (˜67%) or duplications (˜7%) of one or more exons, or by point mutations (˜25%) in the 2.24 Mb dystrophin gene, resulting in the absence of functional dystrophin. BMD is also caused by mutations in the dystrophin gene, but these maintain the open reading frame, yield semi-functional dystrophin proteins, and result in a typically much milder phenotype and longer lifespan.

To date, four AONs have been approved for treatment of DMD: eteplirsen, golodirsen, casimersen and viltolarsen. However, these drugs possess limited efficacy and are each approved for treating only a small percentage of DMD patients.

Thus, there is a continuing need for exon skipping AONs for use in compositions and methods of treating DMD.

### SUMMARY

Provided herein are AONs for use in compositions and methods for treating DMD. The AONs provided herein are reverse complementary to a portion of exon 51 of human dystrophin pre-mRNA. In one embodiment, the AONs provided herein contain at least one modification, as defined herein. In another embodiment, the AONs provided herein are 2′-O-methoxyethyl (“2′-MOE”) RNA oligonucleotides having a phosphorothioate backbone. In another embodiment, the AONs provided herein are 2′-O-methyl (“2′-OMe”) RNA oligonucleotides having a phosphorothioate backbone. In another embodiment, the AONs provided herein are 2′-MOE RNA oligonucleotides having a phosphorothioate backbone, where all cytosines are replaced with 5-methylcytosine and all uracils are replaced with thymine. In another embodiment, the AONs provided herein are 2′-OMe RNA oligonucleotides having a phosphorothioate backbone, where all cytosines are replaced with 5-methylcytosine and all uracils are replaced with thymine.

Also provided herein are methods of treating DMD by administering an AON or composition provided herein. Also provided herein are methods of delaying the onset of DMD by administering an AON or composition provided herein.

### BRIEF DESCRIPTION OF THE DRAWINGS

FIG. 1 shows exon 51 skipping for AONs provided herein.

FIG. 2 shows immunofluorescence analysis of dystrophin expression for AONs provided herein.

FIGS. 3A-B shows in vivo exon skipping (FIG. 3A) and dystrophin levels (FIG. 3B) in quadriceps and heart in hDMDde152/mdx mice for AONs provided herein.

### DETAILED DESCRIPTION

### I. Definitions

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The singular forms “a,” “an,” and “the” include plural references, unless the context clearly dictates otherwise.

As used herein “subject” is an animal, such as a mammal, including human, such as a patient.

As used herein, biological activity refers to the in vivo activities of a compound or physiological responses that result upon in vivo administration of a compound, composition or other mixture. Biological activity, thus, encompasses therapeutic effects and pharmacokinetic behavior of such compounds, compositions and mixtures. Biological activities can be observed in in vitro systems designed to test for such activities.

As used herein, pharmaceutically acceptable derivatives of a compound include, but are not limited to, salts, esters, enol ethers, enol esters, acetals, ketals, orthoesters, hemiacetals, hemiketals, acids, bases, clathrates, solvates or hydrates thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating DMD.

As used herein, amelioration of the symptoms of a particular disorder by administration of a particular compound or pharmaceutical composition refers to any lessening, whether permanent or temporary, lasting or transient that can be attributed to or associated with administration of the compound or pharmaceutical composition.

As used herein, and unless otherwise indicated, the terms “manage,” “managing” and “management” encompass preventing the recurrence of the specified disease or disorder in a subject who has already suffered from the disease or disorder, and/or lengthening the time that a subject who has suffered from the disease or disorder remains in remission. The terms encompass modulating the threshold, development and/or duration of the disease or disorder, or changing the way that a subject responds to the disease or disorder.

Where moieties are specified by their conventional chemical formulae, written from left to right, they equally encompass the chemically identical moieties that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain saturated hydrocarbon radical, which can include di- and multivalent radicals, having the number of carbon atoms designated (i.e., C₁-C₁₀ means one to ten carbons). Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

The term “alkenyl,” by itself or as part of another substituent, means, unless otherwise stated, a straight (i.e., unbranched) or branched chain hydrocarbon radical having one or more carbon-carbon double bonds, which can include di- and multivalent radicals, having the number of carbon atoms designated (i.e., C₁-C₁₀ means one to ten carbons). Examples of alkenyl groups include, but are not limited to, vinyl (i.e., ethenyl), 2-propenyl, crotyl, 2-isopentenyl, 2-(butadienyl), 2,4-pentadienyl, 3-(1,4-pentadienyl), and the higher homologs and isomers.

The term “alkynyl,” by itself or as part of another substituent, means, unless otherwise stated, a straight (i.e., unbranched) or branched chain hydrocarbon radical having one or more carbon-carbon triple bonds, which can include di- and multivalent radicals, having the number of carbon atoms designated (i.e., C₁-C₁₀ means one to ten carbons). Examples of alkynyl groups include, but are not limited to, ethynyl, 1- and 3-propynyl, 3-butynyl, and the higher homologs and isomers.

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, including those groups having 10 or fewer carbon atoms. A “lower alkyl” or “lower alkylene” is a shorter chain alkyl or alkylene group, generally having six or fewer carbon atoms.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain hydrocarbon radical, consisting of a heteroatom selected from the group consisting of O, N, P, Si and S, and wherein the nitrogen and sulfur atoms may optionally be oxidized and the nitrogen atom may have an alkyl substituent to fulfill valency and/or may optionally be quaternized. The heteroatom(s) O, N, P, Si and S may be placed at any interior position of the heteroalkyl group. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For alkylene and heteroalkylene linking groups, no orientation of the linking group is implied by the direction in which the formula of the linking group is written. For example, the formula —C(O)₂R′— represents both —C(O)₂R′- and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively, including bicyclic, tricyclic and bridged bicyclic groups. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, norbornanyl, bicyclo[2.2.2]octanyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, 1- or 2-azabicyclo[2.2.2]octanyl, and the like.

The terms “halo,” by itself or as part of another substituent, means, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (in one embodiment from 1 to 3 rings) which are fused together or linked covalently. The term “heteroaryl” refers to aryl groups that contain from one to four heteroatoms selected from N, O, and S in the ring(s), wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituent moieties for aryl and heteroaryl ring systems may be selected from the group of acceptable substituent moieties described herein. The term “heteroarylium” refers to a heteroaryl group that is positively charged on one or more of the heteroatoms.

The term “oxo” as used herein means an oxygen atom that is double bonded to a carbon atom.

Each of the above terms (e.g., “alkyl,” “heteroalkyl,” “aryl” and “heteroaryl”) are meant to include both substituted and unsubstituted forms of the indicated radical. Non-limiting examples of substituent moieties for each type of radical are provided below.

Substituent moieties for alkyl, heteroalkyl, alkylene, alkenyl, heteroalkylene, heteroalkenyl, alkynyl, cycloalkyl, heterocycloalkyl, cycloalkenyl, and heterocycloalkenyl groups are, in one embodiment, selected from, deuterium, —OR′, ═O, ═NR′, ═N—OR′, —NR′R″, —SR′, halo, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NRSO₂NR′R″, —CN and —NO₂ in a number ranging from zero to the number of hydrogen atoms in such radical. In one embodiment, substituent moieties for cycloalkyl, heterocycloalkyl, cycloalkenyl, and heterocycloalkenyl groups also include substituted and unsubstituted alkyl, substituted and unsubstituted alkenyl, and substituted and unsubstituted alkynyl. R′, R″, R′″ and R″″ each in one embodiment independently are hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound provided herein includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituent moieties, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Substituent moieties for aryl and heteroaryl groups are, in one embodiment, selected from deuterium, halo, substituted and unsubstituted alkyl, substituted and unsubstituted alkenyl, and substituted and unsubstituted alkynyl, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of hydrogens on the aromatic ring system; and where R′, R″, R′″ and R″″ are, in one embodiment, independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound provided herein includes more than one R group, for example, each of the R groups is independently selected as are each R′, R″, R′″ and R″″ groups when more than one of these groups is present.

Two of the substituent moieties on adjacent atoms of an aryl or heteroaryl ring may optionally form a ring of the formula -Q′-C(O)—(CRR′)_{q}-Q″-, wherein Q′ and Q″ are independently —NR—, —O—, —CRR′— or a single bond, and q is an integer of from 0 to 3. Alternatively, two of the substituent moieties on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)ₛ—X′—(CR″R′″)_{d}—, where s and d are independently integers of from 0 to 3, and X′ is —O—, —NR′—, —S—, —S(O)—, —S(O)₂—, or —S(O)₂NR′—. The substituent moieties R, R′, R″ and R′″ are, in one embodiment, independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

As used herein, a prodrug is a compound that upon in vivo administration is metabolized, or otherwise undergoes chemical changes under physiological conditions, by one or more steps or processes or otherwise converted to a biologically, pharmaceutically or therapeutically active form of the compound. Additionally, prodrugs can be converted to a biologically, pharmaceutically or therapeutically active form of the compound by chemical or biochemical methods in an ex vivo environment. For example, prodrugs can be converted to the compounds of the present invention when placed in a transdermal patch reservoir with a suitable enzyme or chemical reagent.

Certain compounds provided herein can exist in unsolvated forms as well as solvated forms, including hydrated forms. In general, the solvated forms are equivalent to unsolvated forms and are encompassed within the scope of the present disclosure. Certain compounds provided herein may exist in multiple crystalline or amorphous forms. In general, all physical forms are equivalent for the uses contemplated herein and are intended to be within the scope of the present disclosure.

Certain compounds provided herein possess asymmetric carbon atoms (optical centers) or double bonds; the racemates, diastereomers, tautomers, geometric isomers and individual isomers are encompassed within the scope of the present disclosure. The compounds provided herein do not include those which are known in the art to be too unstable to synthesize and/or isolate.

The compounds provided herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-¹⁴ (HC). All isotopic variations of the compounds provided herein, whether radioactive or not, are encompassed within the scope of the present disclosure.

### II. Antisense Oligonucleotides for Use in Compositions and Methods

In one embodiment, provided herein are AONs that are reverse complementary to a portion of exon 51 of human dystrophin pre-mRNA. In one embodiment, the AONs provided herein have or contain one of sequences shown in Table 1:

| AON# | |
|---|---|
| (SEQ ID NO.) | Bases (5′ to 3′) |
| 1 | AAAGAAGAAAAAGAAAAA |
| 2 | GAAAAAAGAAGAAAAAGA |
| 3 | AAAAGGAAAAAAGAAGAA |
| 4 | TTGCAAAAAGGAAAAAAG |
| 5 | GGTTTTTGCAAAAAGGAA |
| 6 | TTTTGGGTTTTTGCAAAA |
| 7 | AAATATTTTGGGTTTTTG |
| 8 | AGCTAAAATATTTTGGGT |
| 9 | GTAGGAGCTAAAATATTT |
| 10 | TCTGAGTAGGAGCTAAAA |
| 11 | AACAGTCTGAGTAGGAGC |
| 12 | AGAGTAACAGTCTGAGTA |
| 13 | TCACCAGAGTAACAGTCT |
| 14 | TTGTGTCACCAGAGTAAC |
| 15 | ACAGGTTGTGTCACCAGA |
| 16 | TAACCACAGGTTGTGTCA |
| 17 | CTTAGTAACCACAGGTTG |
| 18 | GTTTCCTTAGTAACCACA |
| 19 | TGGCAGTTTCCTTAGTAA |
| 20 | GGAGATGGCAGTTTCCTT |
| 21 | AGTTTGGAGATGGCAGTT |
| 22 | TTTCTAGTTTGGAGATGG |
| 23 | TGGCATTTCTAGTTTGGA |
| 24 | GAAGATGGCATTTCTAGT |
| 25 | TCAAGGAAGATGGCATTT |
| 26 | CAACATCAAGGAAGATGG |
| 27 | ACCTCCAACATCAAGGAA |
| 28 | CAGGTACCTCCAACATCA |
| 29 | CAGAGCAGGTACCTCCAA |
| 30 | TCTGCCAGAGCAGGTACC |
| 31 | TGAAATCTGCCAGAGCAG |
| 32 | CCGGTTGAAATCTGCCAG |
| 33 | CAAGCCCGGTTGAAATCT |
| 34 | CTGTCCAAGCCCGGTTGA |
| 35 | AAGTTCTGTCCAAGCCCG |
| 36 | TCGGTAAGTTCTGTCCAA |
| 37 | GCCAGTCGGTAAGTTCTG |
| 38 | AGAAAGCCAGTCGGTAAG |
| 39 | AGCAGAGAAAGCCAGTCG |
| 40 | GATCAAGCAGAGAAAGCC |
| 41 | AACTTGATCAAGCAGAGA |
| 42 | TTTATAACTTGATCAAGC |
| 43 | GTGATTTTATAACTTGAT |
| 44 | CCTCTGTGATTTTATAAC |
| 45 | ATCACCCTCTGTGATTTT |
| 46 | CCACCATCACCCTCTGTG |
| 47 | GTCACCCACCATCACCCT |
| 48 | TCAAGGTCACCCACCATC |
| 49 | TATCCTCAAGGTCACCCA |
| 50 | GTTGATATCCTCAAGGTC |
| 51 | ATCTCGTTGATATCCTCA |
| 52 | TGATCATCTCGTTGATAT |
| 53 | CTTGATGATCATCTCGTT |
| 54 | TTCTGCTTGATGATCATC |
| 55 | ATACCTTCTGCTTGATGA |
| 56 | TTCTCATACCTTCTGCTT |
| 57 | ATTTTTTCTCATACCTTC |
| 58 | TTATCATTTTTTCTCATA |
| 59 | AACTTTTATCATTTTTTC |
| 60 | CTGCCAACTTTTATCATT |
| 61 | AACTTCTGCCAACTTTTA |
| 62 | AGAAAAACTTCTGCCAAC |
| 63 | TTTAAAGAAAAACTTCTG |
| 64 | TTCATTTTAAAGAAAAAC |
| 65 | CCCGGTTGAAATCTGCCA |
| 66 | GCCCGGTTGAAATCTGCC |
| 67 | AGCCCGGTTGAAATCTGC |
| 68 | AAGCCCGGTTGAAATCTG |
| 69 | CCAAGCCCGGTTGAAATC |
| 70 | TCCAAGCCCGGTTGAAAT |
| 71 | GTCCAAGCCCGGTTGAAA |
| 72 | TGTCCAAGCCCGGTTGAA |
| 73 | TCTGTCCAAGCCCGGTTG |
| 74 | TTCTGTCCAAGCCCGGTT |
| 75 | GTTCTGTCCAAGCCCGGT |
| 76 | AGTTCTGTCCAAGCCCGG |
| 77 | TAAGTTCTGTCCAAGCCC |
| 78 | GTAAGTTCTGTCCAAGCC |
| 79 | GGTAAGTTCTGTCCAAGC |
| 80 | CGGTAAGTTCTGTCCAAG |
| 81 | GTCGGTAAGTTCTGTCCA |
| 82 | AGTCGGTAAGTTCTGTCC |
| 83 | CAGTCGGTAAGTTCTGTC |
| 84 | CCAGTCGGTAAGTTCTGT |
| 85 | AGCCAGTCGGTAAGTTCT |
| 86 | AAGCCAGTCGGTAAGTTC |
| 87 | AAAGCCAGTCGGTAAGTT |
| 88 | GAAAGCCAGTCGGTAAGT |
| 89 | GAGAAAGCCAGTCGGTAA |
| 90 | AGAGAAAGCCAGTCGGTA |
| 91 | CAGAGAAAGCCAGTCGGT |
| 92 | GCAGAGAAAGCCAGTCGG |
| 93 | AAGCAGAGAAAGCCAGTC |

In another embodiment, the AONs provided herein have or contain one of the sequences provided herein where all nucleotides are RNA. In another embodiment, the AONs provided herein have or contain one of the sequences provided herein where all nucleotides are DNA. In another embodiment, the AONs provided herein have or contain one of the sequences provided herein where the nucleotides are a mixture of RNA and DNA.

As is known to those of skill in the art, naturally occurring oligonucleotides usually contain nucleotides, which contain a sugar moiety and a base moiety, which are linked by a phosphodiester backbone. In one embodiment, the AONs provided herein contain a modification. The modification is a chemical modification of either the sugar moiety or the base moiety of one or more nucleotides in the AON or is a chemical modification of the phosphodiester backbone. Each modification may be independently selected. Thus, an AON provided herein may have two or more distinct modifications.

In one embodiment, the modification is a chemical modification of the sugar moiety of one or more nucleotides in the AON. In another embodiment, the modification is a chemical modification of the sugar moiety of 1, 2, 3, 4 or all nucleotides in the AON.

In one embodiment, the modified sugar moiety for use in the AONs provided herein is a 2′-O-modified RNA such as 2′-O-alkyl or 2′-O-(substituted)alkyl, e.g. 2′-O-methyl, 2′-O-(2-cyanoethyl), 2′-O-(2-methoxy)ethyl (2′-MOE), 2′-O-(2-thiomethyl)ethyl, 2′-O-butyryl, 2′-0-propargyl, 2′-O-acetalester (such as e.g. Biscans et al. Bioorg. Med. Chem. 2015, 23, 5360), 2′-O-allyl, 2′-O-(2S-methoxypropyl), 2′-O—(N-(aminoethyl)carbamoyl)methyl) (2′-AECM), 2′-O-(2-carboxyethyl) and carbamoyl derivatives (Yamada et al. Org. Biomol. Chem. 2014, 12, 6457), 2′-O-(2-amino)propyl, 2′-O-(2-(dimethylamino)propyl), 2′-O-(2-amino)ethyl, 2′-O-(2-(dimethylamino)ethyl), 2′-O-(haloalkoxy)methyl (Arai K. et al. Bioorg. Med. Chem. 2011, 21, 6285), e.g. 2′-O-(2-chloroethoxy)methyl (MCEM), 2′-O-(2,2-dichloroethoxy)methyl (DCEM); 2′-O-alkoxycarbonyl e.g. 2′-O[2-(methoxycarbonyl)ethyl] (MOCE), 2′-O-[2-(N-methylcarbamoyl)ethyl] (MCE), 2′-O-[2-(N,N-dimethylcarbamoyl)ethyl] (DCME), 2′-O[2-(methylthio)ethyl] (2′-MTE), 2′-(ω-O-serinol); 2′-halo e.g. 2′-F, FANA (2′-F arabinosyl nucleic acid); 2′,4′-difluoro-2′-deoxy; carbasugar and azasugar modifications; 3′-O-substituted e.g. 3′-O-methyl, 3′-O-butyryl, 3′-O-propargyl; 4′-substituted e.g. 4′-aminomethyl-2′-O-methyl or 4′-aminomethyl-2′-fluoro; 5′-substituted e.g. 5′-methyl or CNA (Ostergaard et al. ACS Chem. Biol. 2014, 22, 6227); and their derivatives.

In one embodiment, the 2′-substituted RNA is 2′-F, 2′-O-methyl, or 2′-O-(2-methoxyethyl)(i.e., 2′-MOE). In another embodiment, the 2′-substituted RNA is a 2′-MOE. In another embodiment, the 2′-substituted RNA is a 2′-OMe.

In one embodiment, the modified sugar moiety for use in the AONs provided herein is a modification that increases binding affinity to target strands, and/or increases melting temperature of the resulting duplex of said first and/or second oligonucleotide with its target, and/or decreases immunostimulatory effects, and/or increases biostability, and/or improves biodistribution and/or intra-tissue distribution, and/or improves cellular uptake and trafficking.

In one embodiment, the modified sugar moiety for use in the AONs provided herein is a bicyclic nucleic acid (BNA) monomer. A BNA is, in one embodiment, a pentose-derived moiety that has been chemically altered to conformationally restrict the pentose ring. Non-limiting examples of BNAs for use in the AONs provided herein are those where a first cycle such as a pentose ring forms a spirane with a further cyclic moiety so that both cycles share only one atom, BNAs where a first cycle such as a pentose cycle is fused to a further cyclic moiety so that both cycles share two adjacent atoms, and BNAs where a first cycle such as a pentose cycle forms a bridged compound through a moiety that is linked to the first cyclic moiety at two non-adjacent atoms. Such non-adjacent atoms are referred to as bridgehead atoms. Bridged compounds comprise multiple cycles, each of which overlap over at least three atoms. A compound with two cycles wherein those cycles overlap over only two atoms is a fused compound instead. In some bridged compounds, the smallest link between two bridgehead atoms is referred to as the bridging moiety, or as the bridge moiety. In other bridged compounds, when one cycle is a characteristic cycle such as the pentose cycle of a nucleotide, the moiety that is not constitutive to that characteristic cycle is referred to as the bridging moiety. It follows that the nomenclature of bridged bicyclic compounds is context dependent.

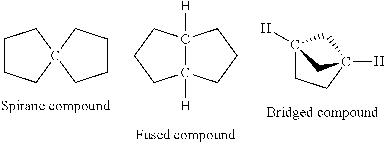

Bicyclic compounds can comprise additional cycles. A bicyclic compound contains at least two cycles, and said two cycles constitute a spirane, a fused system, or a bridged system, or a combination thereof. In one embodiment, bicyclic compounds are fused and bridged compounds. In some embodiments, a BNA is a bridged nucleic acid monomer.

In one embodiment, provided is an AON wherein each occurrence of the BNA in the AON is independently chosen from the group consisting of a conformationally restricted nucleotide (CRN) monomer, a locked nucleic acid (LNA) monomer, a xylo-LNA monomer, an α-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted-2′-amino-LNA monomer, a 2′-thio-LNA monomer, a (2′-0,4′-C) constrained ethyl (cEt) BNA monomer, a (2′-0,4′-C) constrained methoxyethyl (cMOE) BNA monomer, a 2′,4′-BNANC(N—H) monomer, a 2′,4′-BNANC(N-Me) monomer, a 2′,4′-BNANC(N-Bn) monomer, an ethylene-bridged nucleic acid (ENA) monomer, a carba LNA (cLNA) monomer, a 3,4-dihydro-2H-pyran nucleic acid (DpNA) monomer, a 2′-C-bridged bicyclic nucleotide (CBBN) monomer, a heterocyclic-bridged BNA monomer, an amido-bridged BNA monomer, an urea-bridged BNA monomer, a sulfonamide-bridged BNA monomer, a bicyclic carbocyclic nucleotide monomer, a TriNA monomer, an α-L-TriNA monomer, a bicyclo DNA (bcDNA) monomer, an F-bcDNA monomer, a tricyclo DNA (tcDNA) monomer, an F-tcDNA monomer, an oxetane nucleotide monomer, a locked PMO monomer derived from 2′-amino-LNA, and derivatives thereof. In one embodiment, more than one distinct scaffold BNA modification can be used in said oligonucleotide. In some embodiments, each occurrence of the BNA is independently chosen from the group consisting of a conformationally restrained nucleotide (CRN) monomer, a locked nucleic acid (LNA) monomer, a xylo-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted-2′-amino-LNA monomer, a (2′-0,4′-C) constrained ethyl (cEt) LNA monomer, a (2′-0,4′-C) constrained methoxyethyl (cMOE) BNA monomer, a 2′,4′-BNANC(N—H) monomer, a 2′,4′-BNANC(N-Me) monomer, an ethylene-bridged nucleic acid (ENA) monomer, a 2′-C-bridged bicyclic nucleotide (CBBN) monomer, and derivatives thereof.

In one embodiment, each occurrence of the BNA is a locked nucleic acid (LNA) monomer. In another embodiment, the AONs provided herein contain LNA and 2′-MOE monomers. In another embodiment, the AONs provided herein contain LNA and 2′-MeO monomers. In another embodiment, the AONs provided herein contain 1-4 LNA monomers. In another embodiment, the AONs provided herein contain 1-3 LNA monomers. In another embodiment, the AONs provided herein contain 1 or 2 LNA monomers. In another embodiment, the AONs provided herein contain 1, 2, 3 or 4 LNA monomers. In another embodiment, the AONs provided herein contain one LNA monomer. In another embodiment, the AONs provided herein contain two LNA monomers. In another embodiment, the AONs provided herein contain three LNA monomers. In another embodiment, the AONs provided herein contain four LNA monomers. In another embodiment, the AONs provided herein contain two LNA monomers at the two 5′ terminal nucleotide positions of the AON and two LNA monomers at the two 3′ terminal nucleotide positions of the AON.

Structural examples of these BNAs are shown below, where B is a nucleotide base (e.g., A, G, T, C, 5-methylcytosine, etc.), X is a variable and represents O, S or NR, where R is H or alkyl, X₂ is a hydroxyl moiety or another 2′-substitution as defined herein, and L is a backbone linkage as described herein. As known to those of skill in the art, the naming of such modifications in the literature is often arbitrary and does not follow a uniform convention—in this application, the names as provided below are intended to refer to the structures provided below. For comparison, the cyclic scaffold of a conventional RNA monomer is shown first. In the structures shown below, monomers are typically depicted as 3′-terminal monomers. When chirality is not indicated, each enantiomer is individually referenced. Heteroatoms comprised in a cyclic moiety can be replaced with other heteroatoms, e.g., N, O or S.

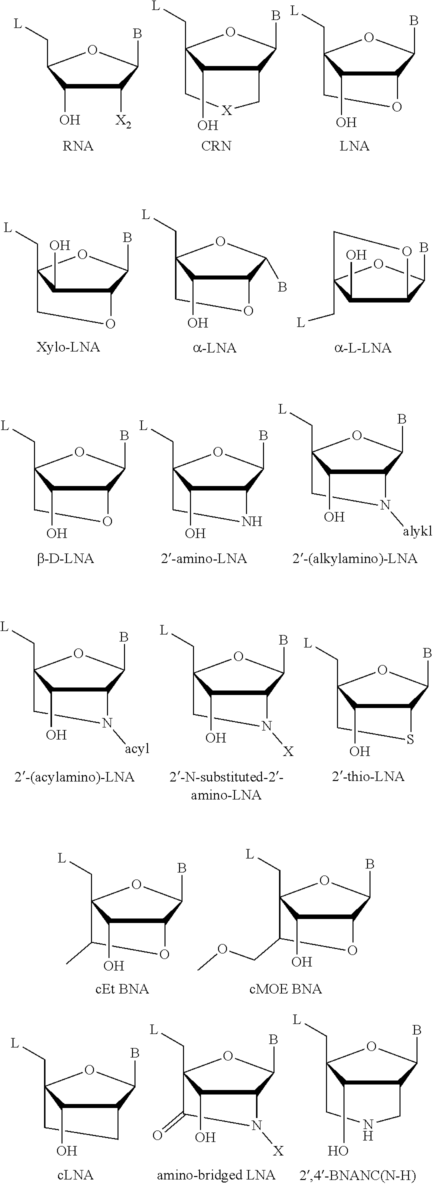

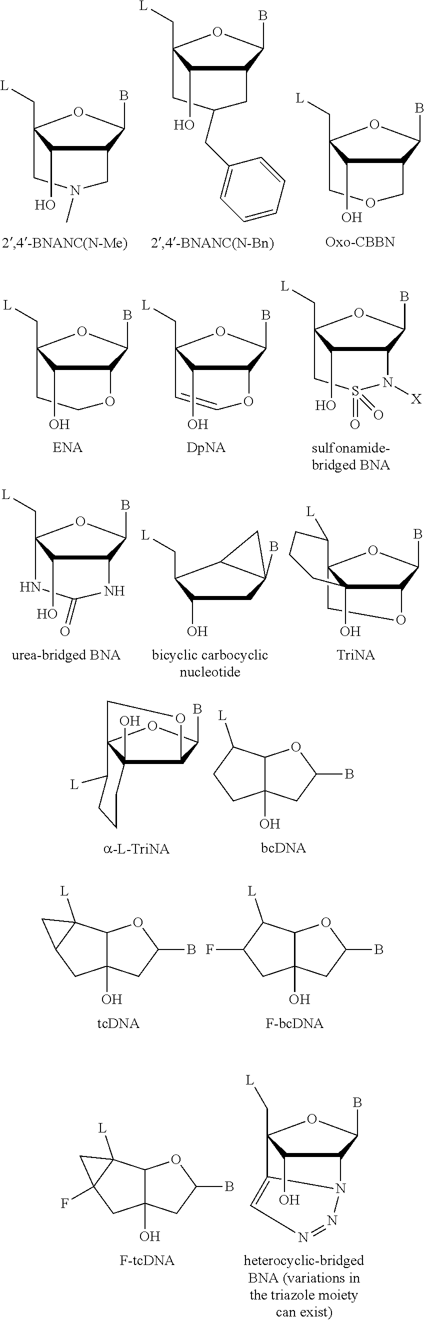

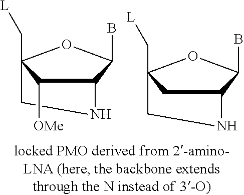

In another embodiment, BNAs for use herein include cEt (2′-0,4′-C constrained ethyl) LNA (doi: 10.1021/ja710342q), cMOE (2′-0,4′-C constrained methoxyethyl) LNA (Seth et al., J. Org. Chem. 2010, 75, 1569-1581), 2′,4′-BNANC(N—H), 2′,4′-BNANC(N-Me), ethylene-bridged nucleic acid (ENA) (doi: 10.1093/nass/1.1.241), carba LNA (cLNA) (doi: 10.1021/jo100170g), DpNA (Osawa et al., J. Org. Chem., 2015, 80 (21), pp 10474-10481), 2′-C-bridged bicyclic nucleotide (CBBN, as in e.g. WO 2014/145356 (MiRagen Therapeutics)), heterocyclic-bridged LNA (as in e.g. WO 2014/126229 (Mitsuoka Y et al.)), amido-bridged LNA (as in e.g. Yamamoto et al. Org. Biomol. Chem. 2015, 13, 3757), urea-bridged LNA (as in e.g. Nishida et al. Chem. Commun. 2010, 46, 5283), sulfonamide-bridged LNA (as in e.g. WO 2014/112463 (Obika S et al.)), bicyclic carbocyclic nucleosides (as in e.g. WO 2015/142910 (Ionis Pharmaceuticals)), TriNA (Hanessian et al., J. Org. Chem., 2013, 78 (18), pp 9064-9075), α-L-TriNA, bicyclo DNA (bcDNA) (Bolli et al., Chem Biol. 1996 March;3(3):197-206), F-bcDNA (DOI: 10.1021/jo402690j), tricyclo DNA (tcDNA) (Murray et al., Nucl. Acids Res., 2012, Vol. 40, No. 13 6135-6143), F-tcDNA (doi: 10.1021/acs.joc.5b00184), or an oxetane nucleotide monomer (Nucleic Acids Res. 2004, 32, 5791-5799). In other embodiments, BNAs for use herein include those disclosed in WO 2011/097641 (ISIS/Ionis Pharmaceuticals) and WO 2016/017422 (Osaka University).

In another embodiment, the chemical modification of the sugar moiety of one or more nucleotides in the AONs provided herein involves replacement of the sugar with another chemical moiety. In these embodiments, the sugar moiety is replaced with, e.g., a morpholine (PMO, PPMO, PMO-X), a peptide derivative (PNA), boron-cluster modified PNA, pyrrolidine-based oxy-peptide nucleic acid (POPNA), glycol- or glycerol-based nucleic acid (GNA), threose-based nucleic acid (TNA), acyclic threoninol-based nucleic acid (aTNA), cationic morpholino-based oligomers (PMOPlus), oligonucleotides with integrated bases and backbones (ONIBs), pyrrolidine-amide oligonucleotides (POMs); and their derivatives.

In one embodiment, the modification is a chemical modification of the base moiety of one or more nucleotides in the AON. In another embodiment, the modification is a chemical modification of the base moiety of 1, 2, 3, 4 or all nucleotides in the AON.

In one embodiment, the AONs provided herein contain at least one modified base moiety. In another embodiment, the AONs provided herein have all base moieties modified. In another embodiment, the AONs provided herein have or contain one of the sequences provided herein where at least one thymine base is a uracil. In another embodiment, the AONs provided herein have or contain one of the sequences provided herein where all thymine bases are uracils. In another embodiment, the AONs provided herein have or contain one of the sequences provided herein where at least one cytosine base is 5-methylcytosine. In another embodiment, the AONs provided herein have or contain one of the sequences provided herein where all cytosine bases are 5-methylcytosine. In another embodiment, the AONs provided herein have or contain one of the sequences provided herein where all thymine bases are uracils and all cytosine bases are 5-methylcytosine.

In one embodiment, the modification is a chemical modification of the backbone of the AON. In another embodiment, the modification is a chemical modification of the backbone of the AON where 1, 2, 3, 4 or all phosphodiester linkages in the AON are modified. In another embodiment, the AON provided herein has at least one phosphorothioate backbone linkage. In another embodiment, the AON provided herein has a fully phosphorothioate backbone linkage. In another embodiment, the AON provided herein has a mixture of phosphorothioate and phosphate backbone linkages. In another embodiment, the AON provided herein has at least one phosphorodiamidate backbone linkage. In another embodiment, the AON provided herein has a fully phosphorodiamidate backbone linkage. In another embodiment, the AON provided herein has a mixture of phosphorodiamidate and phosphate backbone linkages.

In another embodiment, the backbone in the AONs provided herein is a chirally pure phosphorothioate, phosphorodithioate (PS2), phosphonoacetate (PACE), phosphonoacetamide (PACA), thiophosphonoacetate, thiophosphonoacetamide, phosphorothioate prodrug, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, and their derivatives. Another modification includes phosphoramidite, phosphoramidate, N3′→P5′ phosphoramidate, phosphordiamidate, phosphorothiodiamidate, sulfamate, dimethylenesulfoxide, sulfonate, triazole, oxalyl, carbamate, methyleneimino (MMI), 3′-S-phosphorothiolate and thioacetamido nucleic acid (TANA); and their derivatives.

In one embodiment, the AONs provided herein contain a hydroxyalkoxy group. Hydroxyalkoxy groups for use in the AONs provided herein comprises or consists of an ethylene glycol monomer, ethylene glycol oligomer or ethylene glycol polymer (also known as polyethylene glycol, PEG).

In another embodiment, a hydroxyalkoxy group, or at least one hydroxyalkoxy group, or all hydroxyalkoxy groups, used in the AON provided herein comprises or consists of an ethylene glycol monomer, ethylene glycol oligomer or ethylene glycol polymer (also known as polyethylene glycol, PEG). In one embodiment, the linkage between the AON and a hydroxyalkoxy group is covalent.

PEGylation, i.e., the attachment of (chemically activated) hydroxyalkoxy groups, including ethylene glycol monomers or chains, is well known to a person skilled in the art. PEGylation can be done at the —OH group of the 5′ terminal monomer and/or the 3′ terminal monomer of a nucleic acid. This can be done directly or through a spacer (e.g. aminoalkyl hydroxyalkoxy group), for example by click chemistry as known by the person skilled in the art.

Also encompassed is the use of modified PEGylation, wherein said (poly)ethylene glycol is chemically modified and/or contains a moiety attached thereto. In this way said hydroxyalkoxy group acquires an additional property as known in the art. For example, said hydroxyalkoxy group may become cleavable or fluorescent. An example of modified PEGylation includes but is not limited to cleavable PEGylation, wherein the linkage is a degradable (cleavable) linkage. Examples include linkages that are responsive to, for example, pH, light, temperature, reductive or oxidative environments, nucleophiles, synthetic reagents, enzymes, proteases, cathepsin, click-to-release reactions or (other) external stimuli.

In one embodiment, the hydroxyalkoxy group is an unmodified ethylene glycol monomer, ethylene glycol oligomer or ethylene glycol polymer. In another embodiment, the hydroxyalkoxy group is a modified ethylene glycol monomer, ethylene glycol oligomer or ethylene glycol polymer (e.g., modified PEG).

In one embodiment, a hydroxyalkoxy group for use in the AONs provided herein comprises or consists of 1, 2, 3, 4, 5, 6, 7, 8, 9, 10, 11, 12, 13, 14, 15, 16, 17, 18, 19 or 20 ethylene glycol monomers. In some embodiments, the hydroxyalkoxy group comprises or consists of 1 to 20, 1 to 16, 1 to 12, 1 to 8, 1 to 6, 2 to 16, 2 to 12, 2 to 8, 2 to 6, 3 to 12, 3 to 8 or 3 to 6 ethylene glycol monomers. In some embodiments, the hydroxyalkoxy group comprises or consists of 1, 2, 3, 4, 5, 6, 7, 8, 9, 10, 11 or 12 ethylene glycol monomers. In some embodiments, the hydroxyalkoxy group comprises or consists of 3, 4, 5 or 6 ethylene glycol monomers. In some embodiments, the hydroxyalkoxy group comprises or consists of 3 or 6 ethylene glycol monomers. In some embodiments, the hydroxyalkoxy group comprises or consists of 3 ethylene glycol monomers.

In one embodiment, the hydroxyalkoxy group is a diethylene glycol, triethylene glycol (TEG), tetraethylene glycol, pentaethylene glycol or hexaethylene glycol (HEG) group. In some embodiments, the hydroxyalkoxy group is TEG or HEG. In some embodiments, the hydroxyalkoxy group is TEG. In some embodiments, the hydroxyalkoxy group is HEG.

In the AONs provided herein, a hydroxyalkoxy group may be attached to the AON using methods well known to those of skill in the art. For example, the 5′- and/or 3′-terminal OH of the AON may be derivatized as a phosphoramidite, chloroformate, chloramidate or thiophosphoramidite, which is then reacted with the hydroxyalkoxy group (e.g., diethylene glycol, triethylene glycol (TEG), tetraethylene glycol, pentaethylene glycol or hexaethylene glycol (HEG)) under standard coupling conditions to provide a hydroxyalkoxylated AON. Alternatively, the OH of the hydroxyalkoxy group (e.g., diethylene glycol, triethylene glycol (TEG), tetraethylene glycol, pentaethylene glycol or hexaethylene glycol (HEG)) is derivatized as a phosphoramidite, chloroformate, chloramidate or thiophosphoramidite, which is then reacted with the 5′- and/or 3′-terminal OH of the AON under standard coupling conditions to provide a hydroxyalkoxylated AON.

In one embodiment, a hydroxyalkoxy group is attached to the AON through a phosphate linker (PO). In another embodiment, the hydroxyalkoxy group is a TEG group and the hydroxyalkoxylated AON is TEG-PO-AON (i.e., HO(CH₂CH₂O)₃—P(O)(OH)-AON), where the TEG-PO is attached to the 5′-OH of the AON. In another embodiment, the hydroxyalkoxy group is a TEG group and the hydroxyalkoxylated AON is TEG-PO-AON, where the TEG-PO is attached to the 3′-OH of the AON. In another embodiment, two TEG groups are attached to the AON, one at the 3′-OH and the other at the 5′-OH, and the hydroxyalkoxylated AON is (TEG-PO)₂-AON.

In another embodiment, a hydroxyalkoxy group is attached to the AON through a phosphorothioate linker (PS). In another embodiment, the hydroxyalkoxy group is a TEG group and the hydroxyalkoxylated AON is TEG-PS-AON (i.e., HO(CH₂CH₂O)₃—P(S)(OH)-AON), where the TEG-PS is attached to the 5′-OH of the AON. In another embodiment, the hydroxyalkoxy group is a TEG group and the hydroxyalkoxylated AON is TEG-PS-AON, where the TEG-PS is attached to the 3′-OH of the AON. In another embodiment, two TEG groups are attached to the AON, one at the 3′-OH and the other at the 5′-OH, and the hydroxyalkoxylated AON is (TEG-PS)₂-AON.

In another embodiment, two TEG groups are attached to the AON, one at the 3′-OH and the other at the 5′-OH, and the hydroxyalkoxylated AON is TEG-PO-AON-PS-TEG; where TEG-PO is at 5′ and TEG-PS is at 3′; or where TEG-PO is at 3′ and TEG-PS is at 5′.

In another embodiment, the hydroxyalkoxy group is a HEG group and the hydroxyalkoxylated AON is HEG-PO-AON (i.e., HO(CH₂CH₂O)₆—P(O)(OH)-AON), where the HEG-PO is attached to the 5′-OH of the AON. In another embodiment, the hydroxyalkoxy group is a HEG group and the hydroxyalkoxylated AON is HEG-PO-AON, where the HEG-PO is attached to the 3′-OH of the AON. In another embodiment, two HEG groups are attached to the AON, one at the 3′-OH and the other at the 5′-OH, and the hydroxyalkoxylated AON is (HEG-PO)₂-AON.

In another embodiment, the hydroxyalkoxy group is a HEG group and the hydroxyalkoxylated AON is HEG-PS-AON (i.e., HO(CH₂CH₂O)₆—P(S)(OH)-AON), where the HEG-PS is attached to the 5′-OH of the AON. In another embodiment, the hydroxyalkoxy group is a HEG group and the hydroxyalkoxylated AON is HEG-PS-AON, where the HEG-PS is attached to the 3′-OH of the AON. In another embodiment, two HEG groups are attached to the AON, one at the 3′-OH and the other at the 5′-OH, and the hydroxyalkoxylated AON is (HEG-PS)₂-AON.

In another embodiment, two HEG groups are attached to the AON, one at the 3′-OH and the other at the 5′-OH, and the hydroxyalkoxylated AON is HEG-PO-AON-PS-HEG; where HEG-PO is at 5′ and HEG-PS is at 3′; or where HEG-PO is at 3′ and HEG-PS is at 5′. In another embodiment, one TEG and one HEG group are attached to the AON, one at the 3′-OH and the other at the 5′-OH, and the hydroxyalkoxylated AON is TEG-PO-AON-PS-HEG; where TEG-PO is at 5′ and HEG-PS is at 3′; or where TEG-PO is at 3′ and HEG-PS is at 5′. In another embodiment, one TEG and one HEG group are attached to the AON, one at the 3′-OH and the other at the 5′-OH, and the hydroxyalkoxylated AON is HEG-PO-AON-PS-TEG; where HEG-PO is at 5′ and TEG-PS is at 3′; or where HEG-PO is at 3′ and TEG-PS is at 5′.

In another embodiment, the AONs provided herein have or contain the sequence of AON #33, 34, 35, 36, 37, 38 or 39. In another embodiment, the AONs provided herein have or contain the sequence of AON #33, 34, 35, 36, 37, 38 or 39 where all thymine bases are replaced with uracil. In another embodiment, the AONs provided herein have or contain the sequence of AON #33, 38 or 39. In another embodiment, the AONs provided herein have or contain the sequence of AON #33, 38 or 39 where all thymine bases are replaced with uracil.

In one embodiment, the AONs provided herein have one of the sequences provided herein where one or two nucleotides are omitted from the 5′ terminus of the AON, or where one or two nucleotides are omitted from the 3′ terminus of the AON, or where one nucleotide is omitted from the 5′ terminus of the AON and one nucleotide is omitted from the 3′ terminus of the AON. Thus, in this embodiment, the AONs provided herein are 16-mers or 17-mers.

In another embodiment, the AONs provided herein contain one of the sequences provided herein or contain a 16-mer or 17-mer as described above, and are 16 to 30 nucleotides in length. In such embodiments, one of skill in the art would readily be able to determine, based on the well-known sequence of human dystrophin exon 51, which nucleotides to add to the 5′ and/or 3′ terminus of the sequence provided herein, and in what order, in order to obtain AONs longer than 18 nucleotides up to 30 nucleotides in length that are 90%, 95%, 96%, 97%, 98%, 99% or 100% reverse complementary to a portion of human dystrophin exon 51. Such longer AONs are within the scope of this disclosure. In another embodiment, the AONs provided herein are 16 to 25 nucleotides in length. In another embodiment, the AONs provided herein are 16 to 20 nucleotides in length. In another embodiment, the AONs provided herein are 16, 17, 18, 19 or 20 nucleotides in length. In another embodiment, the AONs provided herein are 18 nucleotides in length.

In another embodiment, provided herein is an AON that has one of the sequences provided herein and is fully 2′-MOE RNA modified, where all cytosines are replaced with 5-methylcytosine, and wherein the backbone is a fully phosphorothioate backbone.

In another embodiment, provided herein is an AON that has one of the sequences provided herein and is fully 2′-OMe RNA modified, where all cytosines are replaced with 5-methylcytosine, and wherein the backbone is a fully phosphorothioate backbone.

In another embodiment, provided herein is an AON that has one of the sequences provided herein, where all cytosines are replaced with 5-methylcytosine, the two 5′ terminal nucleotides of the AON are LNA, the two 3′ terminal nucleotides of the AON are LNA, the remaining nucleotides are 2′-MOE RNA modified, and wherein the backbone is a fully phosphorothioate backbone.

In another embodiment, provided herein is an AON that has one of the sequences provided herein, where all cytosines are replaced with 5-methylcytosine, the two 5′ terminal nucleotides of the AON are LNA, the two 3′ terminal nucleotides of the AON are LNA, the remaining nucleotides are 2′-OMe RNA modified, and wherein the backbone is a fully phosphorothioate backbone.

The AONs provided herein may be synthesized using standard solid phase oligonucleotide synthesis techniques. For example, the AONs may be synthesized using either an OP-10 synthesizer (GE/AKTA Oligopilot) or a MerMade 12 Synthesizer (BioAutomation), through standard phosphoramidite protocols. The AONs may be cleaved and deprotected in a two-step sequence (e.g., DEA followed by conc. NH₄OH treatment), purified by anion-exchange chromatography, desalted by size exclusion chromatography and lyophilized.

### III. Pharmaceutical Compositions

The pharmaceutical compositions provided herein contain therapeutically effective amounts of one or more of the AONs provided herein and a pharmaceutically acceptable carrier, diluent or excipient.

The AONs can be formulated into suitable pharmaceutical preparations such as solutions, suspensions, powders, in sterile solutions or suspensions for ophthalmic or parenteral administration, as well as transdermal patch preparation. Typically, the AONs described herein are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In certain embodiments, the concentrations of the AONs in the compositions are effective for delivery of an amount, upon administration, that treats, prevents, or ameliorates one or more of the symptoms and/or progression of a disease or disorder disclosed herein.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the AONs provided herein include any such carriers known to those skilled in the art to be suitable for the particular mode of administration.

In addition, the AONs may be formulated as the sole pharmaceutically active ingredient in the composition or may be combined with other active ingredients. Liposomal suspensions, including tissue-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the subject treated. The therapeutically effective concentration may be determined empirically by testing the AONs in in vitro and in vivo systems described herein and then extrapolated therefrom for dosages for humans. In some embodiments, the AON is administered in a method to achieve a therapeutically effective concentration of the drug. In some embodiments, a companion diagnostic (see, e.g., Olsen D and Jorgensen J T, Front. Oncol., 2014 May 16, 4:105, doi: 10.3389/fonC.2014.00105) is used to determine the therapeutic concentration and safety profile of the active compound in specific subjects or subject populations.

The concentration of AON in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of a disease or disorder disclosed herein.

In certain embodiments, a therapeutically effective dosage should produce a serum concentration of active ingredient of from about 0.1 ng/mL to about 50-100 μg/mL. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The AON may be administered at once or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the AONs described herein or pharmaceutically acceptable salts thereof are mixed with a suitable pharmaceutical carrier or vehicle for systemic, topical or local administration to form pharmaceutical compositions. AONs are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to parenteral, subcutaneous, intravenous, intramuscular, intraperitoneal, intrathecal, dermal, transdermal or buccal.

Solutions or suspensions used for parenteral, intradermal or subcutaneous application can include any of the following components: a sterile diluent, such as water for injection, saline solution, fixed oil, polyethylene glycol, glycerin, propylene glycol, dimethyl acetamide or other synthetic solvent; antimicrobial agents, such as benzyl alcohol and methyl parabens; antioxidants, such as ascorbic acid and sodium bisulfate; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the AONs exhibit insufficient solubility, methods for solubilizing AONs may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the AON(s), the resulting mixture may be a solution, suspension, emulsion or the like. The form of the resulting mixture depends upon a number of factors, including the intended mode of administration and the solubility of the AON in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as powders, granules, sterile parenteral solutions or suspensions, and oil water emulsions containing suitable quantities of the AONs or pharmaceutically acceptable salts thereof. The pharmaceutically therapeutically active AONs and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non-toxic carrier may be prepared. Such compositions include solutions, suspensions, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, in certain embodiments, about 0.1 85% or about 75-95%.

The active AONs or pharmaceutically acceptable salts may be prepared with carriers that protect the compound against rapid elimination from the body, such as time release formulations or coatings.

The compositions may include other active AONs to obtain desired combinations of properties. The AONs provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

### A. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. In some embodiments, the suspension is a suspension of microparticles or nanoparticles. In some embodiments, the emulsion is an emulsion of microparticles or nanoparticles. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, an AON provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The AON diffuses through the outer polymeric membrane in a release rate controlling step. The percentage of active AON contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active AON is adjusted so that an injection provides an effective amount to produce the desired pharmacological effect. The exact dose depends on the age, weight and condition of the subject or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active AON is an effective mode of administration. Another embodiment is a sterile aqueous or oily solution or suspension containing an active AON injected as necessary to produce the desired pharmacological effect.

Injectables are designed for local and systemic administration. Typically, a therapeutically effective dosage is formulated to contain a concentration of at least about 0.1% w/w up to about 90% w/w or more, such as more than 1% w/w of the active AON to the treated tissue(s). The active AON may be administered at once or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The AON may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### B. Lyophilized Powders

Also provided herein are lyophilized powders, which can be reconstituted for administration as solutions, emulsions and other mixtures. They may also be reconstituted and formulated as solids or gels.

The sterile, lyophilized powder is prepared by dissolving an AON provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

### C. Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

### D. Sustained Release Compositions

AONs provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased subject compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an AON can be stimulated by various conditions including, but not limited to, pH, temperature, enzymes, water, or other physiological conditions or compounds.

In certain embodiments, the AON may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

Other controlled release systems are discussed in the review by Langer (Science 249:1527-1533 (1990). The AON can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The AON then diffuses through the outer polymeric membrane in a release rate controlling step. The percentage of active ingredient contained in such parenteral compositions is highly dependent on the specific nature thereof, as well as the needs of the subject.

### E. Targeted Formulations

The AONs provided herein, or pharmaceutically acceptable salts thereof, may also be formulated to be targeted to a particular tissue, receptor, or other area of the body of the subject to be treated, including liposome-, resealed erythrocyte-, and antibody-based delivery systems. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, the antibody-based delivery system is an antibody-drug conjugate (“ADC”), e.g., as described in Hamilton G S, Biologicals, 2015 September, 43(5):318-32; Kim E G and Kim K M, Biomol. Ther. (Seoul), 2015 November, 23(6):493-509; and Peters C and Brown S, Biosci. Rep., 2015 Jun. 12, 35(4) pii: e00225, each of which is incorporated herein by reference.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of an AON provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### F. Articles of Manufacture

The AONs or pharmaceutically acceptable salts can be packaged as articles of manufacture containing packaging material, an AON or pharmaceutically acceptable salt thereof provided herein, which is used for treatment, prevention or amelioration of one or more symptoms or progression of a disease or disorder disclosed herein, and a label that indicates that the compound or pharmaceutically acceptable salt thereof is used for treatment, prevention or amelioration of one or more symptoms or progression of a disease or disorder disclosed herein.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the AONs and compositions provided herein are contemplated.

In certain embodiments, provided herein also are kits which, when used by the medical practitioner, can simplify the administration of appropriate amounts of AONs to a subject. In certain embodiments, the kit provided herein includes a container and a dosage form of an AON provided herein or a pharmaceutically acceptable salt, solvate, or prodrug thereof.

In certain embodiments, the kit includes a container comprising a dosage form of the AON provided herein or a pharmaceutically acceptable salt, solvate, or prodrug thereof, in a container comprising one or more other therapeutic agent(s) described herein.

Kits provided herein can further include devices that are used to administer the AONs. Examples of such devices include, but are not limited to, syringes, needle-less injectors drip bags, patches, and inhalers.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### IV. Dosing

The AONs and pharmaceutical compositions provided herein may be dosed in certain therapeutically or prophylactically effective amounts, certain time intervals, certain dosage forms, and certain dosage administration methods as described below.

In certain embodiments, a therapeutically or prophylactically effective amount of the AON is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, from about 0.05 to about 10 mg per day, from about 0.05 to about 5 mg per day, from about 0.1 to about 5 mg per day, or from about 0.5 to about 5 mg per day.

In certain embodiments, the therapeutically or prophylactically effective amount is about 0.1, about 0.2, about 0.5, about 1, about 2, about 3, about 4, about 5, about 6, about 7, about 8, about 9, about 10, about 15, about 20, about 25, about 30, about 40, about 45, about 50, about 60, about 70, about 80, about 90, about 100, or about 150 mg per day.

In one embodiment, the recommended daily dose range of the AON provided herein, or a derivative thereof, for the conditions described herein lie within the range of from about 0.5 mg to about 50 mg per day, in one embodiment given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day. In a specific embodiment, the AON can be administered in an amount of about 25 mg/day. In a particular embodiment, the AON can be administered in an amount of about 10 mg/day. In a particular embodiment, the AON can be administered in an amount of about 5 mg/day. In a particular embodiment, the AON can be administered in an amount of about 4 mg/day. In a particular embodiment, the AON can be administered in an amount of about 3 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In certain embodiments, the amount of the AON administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the AON administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM.

As used herein, the term “plasma concentration at steady state” is the concentration reached after a period of administration of a compound provided herein, or a derivative thereof. Once steady state is reached, there are minor peaks and troughs on the time dependent curve of the plasma concentration of the compound.

In certain embodiments, the amount of the AON administered is sufficient to provide a maximum plasma concentration (peak concentration) of the compound, ranging from about 0.001 to about 50 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In certain embodiments, the amount of the AON administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In certain embodiments, the amount of the AON administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

The methods provided herein encompass treating a patient regardless of subject's age, although some diseases or disorders are more common in certain age groups.

Depending on the disease to be treated and the subject's condition, the AON provided herein, or a derivative thereof, may be administered by parenteral (e.g., intramuscular, intraperitoneal, intravenous, CIV, intracisternal injection or infusion, subcutaneous injection, or implant), or topical (e.g., transdermal or local) routes of administration. The AON provided herein, or a derivative thereof, may be formulated, alone or together, in suitable dosage unit with pharmaceutically acceptable excipients, carriers, adjuvants and vehicles, appropriate for each route of administration.

In another embodiment, the AON provided herein, or a derivative thereof, is administered parenterally. In yet another embodiment, the AON provided herein, or a derivative thereof, is administered intravenously.

The AON provided herein, or a derivative thereof, can be delivered as a single dose such as, e.g., a single bolus injection, or over time, such as, e.g., continuous infusion over time or divided bolus doses over time. The AON can be administered repeatedly if necessary, for example, until the subject experiences stable disease or regression, or until the subject experiences disease progression or unacceptable toxicity.

The AON provided herein, or a derivative thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as the compound provided herein, or a derivative thereof, is administered once or more than once each day, for example, for a period of time. The term “continuous” is intended to mean that a therapeutic compound, such as the compound provided herein or a derivative thereof, is administered daily for an uninterrupted period of at least 10 days to 52 weeks. The term “intermittent” or “intermittently” as used herein is intended to mean stopping and starting at either regular or irregular intervals. For example, intermittent administration of the AON provided herein or a derivative thereof is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the compound provided herein or a derivative thereof, is administered daily or continuously but with a rest period. In some such embodiments, administration is once a day for two to six days, then a rest period with no administration for five to seven days.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein, or a derivative thereof, is administered once a day. In another embodiment, the AON provided herein, or a derivative thereof, is administered twice a day. In yet another embodiment, the AON provided herein, or a derivative thereof, is administered three times a day. In still another embodiment, the AON provided herein, or a derivative thereof, is administered four times a day.

In certain embodiments, the AON provided herein, or a derivative thereof, is administered once per day from one day to six months, from one week to three months, from one week to four weeks, from one week to three weeks, or from one week to two weeks. In certain embodiments, the AON provided herein, or a derivative thereof, is administered once per day for one week, two weeks, three weeks, or four weeks. In one embodiment, the AON provided herein, or a derivative thereof, is administered once per day for 4 days. In one embodiment, the AON provided herein, or a derivative thereof, is administered once per day for 5 days. In one embodiment, the AON provided herein, or a derivative thereof, is administered once per day for 6 days. In one embodiment, the AON provided herein, or a derivative thereof, is administered once per day for one week. In another embodiment, the AON provided herein, or a derivative thereof, is administered once per day for two weeks. In yet another embodiment, the AON provided herein, or a derivative thereof, is administered once per day for three weeks. In still another embodiment, the AON provided herein, or a derivative thereof, is administered once per day for four weeks.

### V. Methods of Treatment

In another embodiment, a method of treating a subject with Duchenne muscular dystrophy (DMD) is provided. In one embodiment, the method involves the step of administering to the subject an AON or composition provided herein. In another embodiment, a method of delaying the onset of DMD by administering an AON or composition provided herein is provided. In another embodiment, the methods alleviate one or more symptom(s) of DMD.

Alleviating one or more symptom(s) of DMD in an individual using an AON provided herein may be assessed by any of the following assays: prolongation of time to loss of walking, improvement of muscle strength, improvement of the ability to lift weight, improvement of the time taken to rise from the floor, improvement in the nine-meter walking time, improvement in the time taken for four-stairs climbing, improvement of the leg function grade, improvement of the pulmonary function, improvement of cardiac function, improvement of the quality of life. Each of these assays is known to the skilled person. For each of these assays, as soon as a detectable improvement or prolongation of a parameter measured in an assay has been found, it will preferably mean that one or more symptoms of DMD has been alleviated in an individual using an AON provided herein. Detectable improvement or prolongation is in one embodiment a statistically significant improvement or prolongation as described in Hodgetts et al. Neuromuscular Disorders 2006; 16: 591-602. Alternatively, the alleviation of one or more symptom(s) of DMD may be assessed by measuring an improvement of a muscle fiber function, integrity and/or survival. In another embodiment, one or more symptom(s) of a DMD patient is/are alleviated and/or one or more characteristic(s) of one or more muscle cells from a DMD patient is/are improved. Such symptoms or characteristics may be assessed at the cellular, tissue level or on the patient self.

An alleviation of one or more characteristics of a muscle cell from a patient may be assessed by any of the following assays on a myogenic cell or muscle cell from a patient: reduced calcium uptake by muscle cells, decreased collagen synthesis, altered morphology, altered lipid biosynthesis, decreased oxidative stress, and/or improved muscle fiber function, integrity, and/or survival. These parameters are usually assessed using immunofluorescence and/or histochemical analyses of cross sections of muscle biopsies.

The improvement of muscle fiber function, integrity and/or survival may be assessed using at least one of the following assays: a detectable decrease of creatine kinase in blood, a detectable decrease of necrosis of muscle fibers in a biopsy cross-section of a muscle suspected to be dystrophic, and/or a detectable increase of the homogeneity of the diameter of muscle fibers in a biopsy cross-section of a muscle suspected to be dystrophic. Each of these assays is known to the skilled person.

Creatine kinase may be detected in blood as described in Hodgetts et al. (2006). A detectable decrease in creatine kinase may mean a decrease of 5%, 10%, 20%, 30%, 40%, 50%, 60%, 70%, 80%, 90% or more compared to the concentration of creatine kinase in a same DMD patient before treatment.

A detectable decrease of necrosis of muscle fibers is preferably assessed in a muscle biopsy, more preferably as described in Hodgetts et al. (2006), using biopsy cross-sections. A detectable decrease of necrosis may be a decrease of 5%, 10%, 20%, 30%, 40%, 50%, 60%, 70%, 80%, 90% or more of the area wherein necrosis has been identified using biopsy cross-sections. The decrease is measured by comparison to the necrosis as assessed in a same DMD patient before treatment.

A detectable increase of the homogeneity of the diameter of a muscle fiber is preferably assessed in a muscle biopsy cross-section, more preferably as described in Hodgetts et al. (supra). The increase is measured by comparison to the homogeneity of the diameter of a muscle fiber in a same DMD patient before treatment.

In one embodiment, an AON provided herein provides said individual with a functional or a semi-functional dystrophin protein, and is able to, for at least in part decrease the production of an aberrant dystrophin protein in said individual.

In one embodiment, providing an individual with a functional or a semi-functional dystrophin protein means an increase in the production of functional or semi-functional dystrophin protein. Increasing the production of functional or semi-functional dystrophin mRNA, or functional or semi-functional dystrophin protein, means a detectable increase or at least 5%, 10%, 20%, 30%, 40%, 50%, 60%, 70%, 80%, 90%, 100%, 120%, 140%, 160%, 180%, 200% or more compared to the initial amount of functional or semi-functional mRNA, or functional or semi-functional dystrophin protein, as detectable by RT-digital droplet PCR (mRNA) (Verheul et al., PLoS ONE 2016, 11(9):e0162467) or immunofluorescence (Beekman et al., PLoS ONE 2014; 9(9): e107494), western blot, or capillary Western immunoassay (Beekman et al., PLoS ONE 2018; 13(4): e0195850) analysis (protein). In another embodiment, said initial amount is the amount of functional or semifunctional mRNA, or functional or semi-functional dystrophin protein, at the onset of inducing exon-skipping in the dystrophin pre-mRNA in a cell, in an organ, in a tissue and/or in an individual using a compound of the invention.

Decreasing the production of an aberrant dystrophin mRNA, or aberrant dystrophin protein, means that 90%, 80%, 70%, 60%, 50%, 40%, 30%, 20%, 10%, 5% or less of the initial amount of aberrant dystrophin mRNA, or aberrant dystrophin protein, is still detectable by RT-digital droplet PCR (mRNA) or immunofluorescence, western blot, or capillary Western immunoassay (Wes) analysis (protein). In one embodiment, said initial amount is the amount of aberrant dystrophin mRNA, or aberrant dystrophin protein, at the onset of inducing exon-skipping in the dystrophin pre-mRNA in a cell, in an organ, in a tissue and/or in an individual using an AON of the invention. An aberrant dystrophin mRNA or protein is also referred to herein as a less functional (compared to a wild type functional dystrophin protein) or a non-functional dystrophin mRNA or protein. A non-functional dystrophin protein is a dystrophin protein which is not able to bind actin and/or members of the DGC protein complex. A non-functional dystrophin protein or dystrophin mRNA does typically not have or does not encode a dystrophin protein with an intact C-terminus of the protein. The detection of a functional or semi-functional dystrophin mRNA or protein may be done as for an aberrant dystrophin mRNA or protein.

Once a DMD patient is provided with a functional or a semi-functional dystrophin protein, at least part of the cause of DMD is taken away. Hence, it would then be expected that the symptoms of DMD are at least partly alleviated, or that the rate with which the symptoms worsen is decreased, resulting in a slower decline. The enhanced skipping frequency also increases the level of functional or a semi-functional dystrophin protein produced in a muscle cell of a DMD individual.

### VI. Combination Therapy with a Second Active Agent

The AON provided herein, or a derivative thereof, can also be combined or used in combination with other therapeutic agents useful in the treatment and/or prevention of DMD.

In one embodiment, provided herein is a method of treating, preventing, or managing DMD, comprising administering to a subject an AON provided herein, or a derivative thereof, in combination with one or more second active agents.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as an AON provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the AON provided herein, or a derivative thereof and one or more second active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream) and the disease or disorder being treated.

The route of administration of the AON provided herein, or a derivative thereof, is independent of the route of administration of a second therapy. In another embodiment, the AON provided herein, or a derivative thereof, is administered intravenously. Thus, in accordance with these embodiments, the AON provided herein, or a derivative thereof, is administered intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, the AON provided herein, or a derivative thereof, and a second therapy are administered by the same mode of administration, e.g., by IV. In another embodiment, the AON provided herein, or a derivative thereof, is administered by one mode of administration, e.g., by IV, whereas the second agent is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount of the AON provided herein, or a derivative thereof, and any optional additional active agents concurrently administered to the subject.

One or more second active ingredients or agents can be used together with the AON provided herein, or a derivative thereof, in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, AONs and monoclonal and polyclonal antibodies. Typical large molecule active agents are biological molecules, such as naturally occurring or synthetic or recombinant proteins. Specific examples for use herein include eteplirsen, casimersen, golodirsen, viltolarsen and SRP-5051 (Sarepta Therapeutics).

Examples of small molecules include corticosteroids, such as deflazacort.

Other therapies that may be combined with the AONs provided herein include gene therapy (e.g., SRP-9001, GALGT2 or GNT 0004 (Sarepta Therapeutics)), gene editing (e.g., CRISPR/CAS9 (Sarepta Therapeutics)) and cellular therapy (e.g., CAP-1002 (Capricor Therapeutics/Nippon Shinyaku Co. Ltd.)).

### VII. Examples

The examples below are meant to illustrate certain embodiments provided herein, and not to limit the scope of this disclosure.

### Example 1

A model system was a KM571 human immortalized myoblast line with an exon 52 deletion. The KM571 cells were previously immortalized using hTERT and CDK4 (PMID: 22040608). On day 1, 138,000 cells per well were nucleofected (Lonza, setting DS-150, SF solution) in the presence of 1.4 micromolar of the individual AONs (2′-MOE (2′-O-methoxyethyl RNA), 5-methylcytosines, thymine (no uracil), and phosphorothioate backbone), then diluted 10-fold with Skeletal Muscle growth medium (PromoCell [cat #: C-23060], final volume 12.5% HI-FBS and 100 units/mL Pen/Strep), and then transferred to a 96-well tissue culture plate. On day 4, the cells were harvested using TaqMan Gene Expression Cells-to-CT (Thermo Fisher Scientific, 55 microliter final lysis volume), 10 microliter of RNA was reverse transcribed, and then 4 microliters of the resulting cDNA were evaluated by qPCR (skip forward primer 5′-AAAGCAGCCTGACCTAGCT-3′ (SEQ ID NO: 94), skip probe 5′-ACCACTATTGGAGCCTTTGAAAGA-3′ (SEQ ID NO: 95), and skip reverse primer 5′-CTTGTACTTCATCCCACTGATTCTGA-3′ (SEQ ID NO: 96)). A double-stranded DNA fragment of the skipped amplicon (IDT, exon 50 spliced to exon 53) was used to validate the assay and to quantify the number of copies in unknown samples (reported as KM571—Skip Copies e50-53). Blank cells in the table were at undetermined background levels. For the KM571 cells, the DMD message was undergoing nonsense mediated decay without treatment. Successful skipping of exon 51 in these cells restored the reading frame, increased the half-life of the mRNA, and allowed for a more complete translation.

Results

Results are shown in Table 2:

KM571- Skip Copies
e50-53

| AON# | Avg | SD |
|---|---|---|
| 1 | | |
| 2 | | |
| 4 | | |
| 5 | 28.8 | 7.8 |
| 9 | | |
| 10 | 422.8 | 31.0 |
| 11 | 242.2 | 72.9 |
| 12 | 27.1 | 12.6 |
| 13 | 261.8 | 19.6 |
| 14 | 253.9 | 21.5 |
| 15 | 585.0 | 41.4 |
| 16 | 131.7 | 16.4 |
| 17 | 2.3 | 0.5 |
| 18 | 1069.7 | 20.3 |
| 19 | 170.3 | 25.8 |
| 20 | 942.3 | 112.8 |
| 21 | 599.9 | 220.7 |
| 22 | 661.6 | 93.1 |
| 23 | 839.7 | 136.1 |
| 24 | 866.7 | 187.6 |
| 25 | 1757.4 | 281.4 |
| 26 | 98.5 | 17.1 |
| 27 | 322.6 | 63.3 |
| 28 | 5.4 | 0.8 |
| 29 | 0.4 | 0.6 |
| 30 | 1.8 | 0.9 |
| 31 | | |
| 32 | | |
| 33 | 6.7 | 2.7 |
| 34 | 4.7 | 2.1 |
| 35 | 7.8 | 1.3 |
| 36 | 1246.8 | 131.2 |
| 37 | 41.5 | 15.0 |
| 38 | 13.0 | 6.5 |
| 39 | 1.8 | 1.0 |
| 40 | 10.5 | 1.8 |
| 51 | 0.8 | 0.8 |
| 52 | 20.0 | 6.4 |
| 55 | 0.9 | 0.9 |
| 56 | 21.7 | 5.1 |
| 57 | 3.1 | 1.9 |
| 58 | 1.1 | 0.5 |

### Example 2

In Vitro Screening of AONs in DMD Patient (Del 48-50) Myotube Cultures

A series of overlapping AONs were screened at 800 nM in DMD patient (del 48-50) myotube cultures. As shown in Table 3 below, AON #79A having the base sequence of AON #79 and 2′-OMe phosphorothioate modification with 5-methylcytosines and 5′ and 3′ LNAs was optimized for LNA content and positions, implementing one or two 5′ terminal guanine-LNA's, a 3′ terminal cytosine-LNA and/or one or two additional internal guanine-LNAs.

TABLE 3
Optimization of LNA content and position in AONs.

| AON # | Chemical Composition |
|---|---|
| 79A | +140L-G, +123L-C |
| 79B | +140L-G, +139L-G, +124L-G, +123L-C |
| 79C | +140L-G, +139L-G, +135L-G, +123L-C |
| 79D | +140L-G, +139L-G, +130L-G, +123L-C |
| 79E | +140L-G, +139L-G, +135L-G, +130L-G, +123L-C |
| 79F | +140L-G, +139L-G, +135L-G, +130L-G, +124L-G, +123L-C |
| 79G | 5′ TEG, +140L-G, +139L-G, +135L-G, +123L-C |
| 80A | +139L-G, +122L-C |
| 80B | +139L-G, +135L-G, +122L-C |

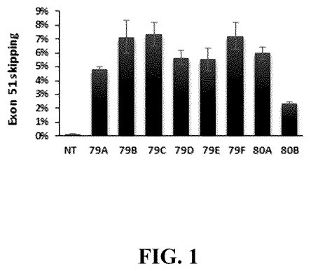

Comparative testing of these AONs at 800 nM resulted in the identification of 3 AONs (AON #79B, AON #79C and AON #79F) with the highest exon 51 skipping efficacy (FIG. 1). Of these AONs with slightly different LNA-profiles, AON #79C was most efficient (7.3%). In subsequent in vitro comparative screening assays AON #79C was found to be 10-fold more effective than drisapersen.

### Example 3

Immunofluorescence Analysis of Dystrophin Expression at the Muscle Fiber Membranes

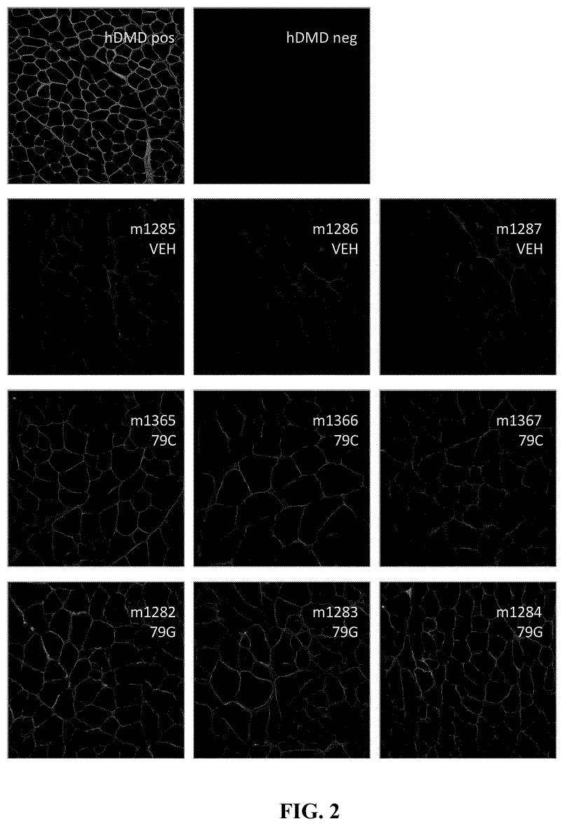

Cryosections (8 μm) from quadriceps muscles were mounted on Superfrost Ultra Plus microscopy slides (Fisher Scientific), and incubated for 2 hr with primary antibody rabbit polyclonal anti dystrophin (Ab15277, Abcam, dilution 1/200). Slides were rinsed and washed twice for 5 minutes in PBS and subsequently incubated for 1 hr with secondary antibody goat anti rabbit AlexaFluor 488 (ThermoFisher) in a dilution of 1/250. Imaging of slides was performed on the same day on a Zeiss LSM 710 confocal microscope using a 25× objective and laser intensity 6%. Shown in FIG. 2 are images from 3 different mice per VEH (vehicle) or AON group. Upper panel: control sections from a non-dystrophic hDMD mouse (pos=with dystrophin primary antibody Ab15277, neg=with rabbit IgG isotype primary antibody Ab27478).

### Example 4

hDMDde152/mdx mice were analyzed after 13 weekly tail vein injections of 18 mg/kg AON #79C or AON #79G. At the doses tested in a study in hDMDde152/mdx mice, AON #79C and AON #79G appeared to be similarly well tolerated, with no effect on survival, body weight, or survival routine clinical chemistry or haematology. In general, no or only minimal histopathological changes were observed in kidney, liver, spleen, lymph nodes, skeletal muscle and heart. In liver hepatocellular single cell necrosis was observed in 6 out of 11 mice given AON #79G and the severity was generally minimal. Additionally, minimal hepatocellular single cell necrosis was observed in one out of 10 mice given AON #79C, but not in any vehicle-control mice of either gender. In the heart, myocardial fibrosis/fibroplasia were observed at a slightly higher incidence and/or severity in treated groups than in the control group. Any relationship between these histopathological changes and AON treatment was unclear, although it could not be excluded. All the other histopathological findings in the heart were observed at similar incidence/severity in control and treated groups and were therefore considered unlikely to be related to AON-treatment. In skeletal muscle, fiber necrosis, muscular atrophy, basophilia/regeneration, inflammation, mineralization and fatty changes were observed as part of the expected morphological appearance of the skeletal muscle in this DMD mouse model (see Table 4 below). After treatment with each of the AONs (AON #79C or AON #79G) there was a reduction of incidence and/or severity of muscle fiber atrophy, necrosis and inflammation compared to vehicle treated mice, which may relate to the restored dystrophin expression.

TABLE 4
Incidence of relevant histological findings in skeletal muscle

| AON | Vehicle | AON#79C | AON#79G |
|---|---|---|---|
| No. of Male/Females | 6/4 | 6/4 | 7/4 |
| Muscle fiber atrophy | | | |
| Minimal | — | 2/1 | 1/2 |
| Mild | —/2 | 3/3 | 4/2 |
| Moderate | 4/1 | — | 2/— |
| Marked | 2/1 | — | — |
| Muscle fiber mineralization | | | |
| Minimal | 5/3 | 4/1 | 2/2 |
| Mild | 1/1 | 1/2 | 5/2 |
| Fatty change | | | |
| Minimal | —/1 | —/2 | 1/3 |
| Muscle fiber necrosis | | | |
| Minimal | 2/2 | 2/— | 4/1 |
| Mild | 3/— | — | — |
| Moderate | —/1 | — | — |
| Marked | —/1 | — | — |
| Basophilia/regeneration | | | |
| Minimal | 3/3 | —/1 | 1/— |
| Moderate | —/1 | — | — |
| Inflammation | | | |
| Minimal | 6/2 | — | —/1 |
| Moderate | —/1 | — | — |

—: finding not observed in mice in the group.

### Example 5

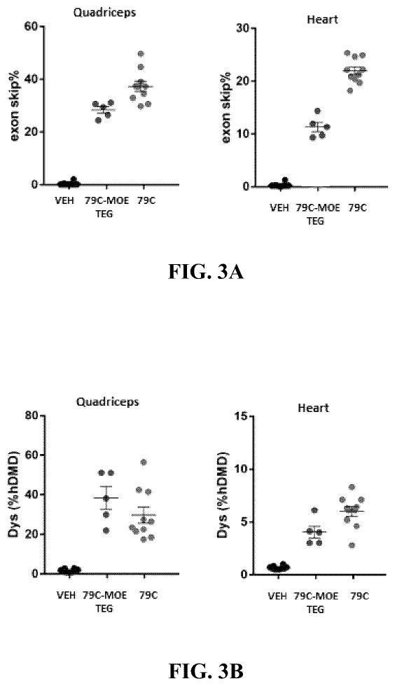

hDMDde152/mdx mice were analyzed after 13 weekly tail vein injections of 18 mg/kg AON #79C or an isosequential AON having 2′-MOE instead of 2′-OMe sugar modification and a 5′-TEG group. Mice were sacrificed 14 days post last dose. All mice in the study survived. Exon skpping and dystrophin levels were similar in quadriceps and heart for both groups. Results are shown in FIG. 3A-B.

### Example 6

AONs provided herein were tested for dystrophin pre-mRNA exon 51 skipping using the assay described in Example 1. The data reported in Tables 5 and 6 are expressed as fold change relative to AON #79C by using an additional qPCR assay for the UBC housekeeping gene and applying the 2^{-ΔΔCT} method. Briefly, the average potency, based on 1-4 potency results, was calculated in relative fold:

Delta ct: skip cts—housekeeping gene cts
Double delta ct: delta cts—average delta cts of no treatment
Fold: 2\hat{ }(double delta cts)
Potency: Fold values relative to fold values of AON #79C

Results

The average potency for the AONs provided herein in inducing dystrophin pre-mRNA exon 51 skipping is shown in the tables below. In Table 5, the AONs tested were fully phosphorothioate backbone, all cytosines are replaced with 5-methylcytosine, and all nucleosides are 2′-MOE RNA nucleosides. In Table 6, the AONs tested were fully phosphorothioate backbone, all cytosines are replaced with 5-methylcytosine, nucleotides 1, 2, 17 and 18 are LNA, and nucleotides 3-16 are 2′-MOE RNA nucleosides.

TABLE 5

| AON# (SEQ ID NO.) | Average Potency |
|---|---|
| 32 | 0.0125 |
| 65 | 0.0175 |
| 66 | 0.0525 |
| 67 | 0.05 |
| 68 | 0.0375 |
| 33 | 0.0825 |
| 69 | 0.15 |
| 70 | 0.2025 |
| 71 | 0.0275 |
| 72 | 0.035 |
| 34 | 0.0675 |
| 73 | 0.0675 |
| 74 | 0.275 |
| 75 | 0.15 |
| 76 | 0.21 |
| 35 | 0.09 |
| 77 | 0.1825 |
| 78 | 0.325 |
| 79 | 0.9975 |
| 80 | 0.9425 |
| 36 | 1.1425 |
| 81 | 1.1075 |
| 82 | 0.73 |
| 83 | 0.655 |
| 84 | 0.6575 |
| 37 | 0.21 |
| 85 | 0.2675 |
| 86 | 0.2475 |
| 87 | 0.1225 |
| 88 | 0.2675 |
| 38 | 0.22 |
| 89 | 0.09 |
| 90 | 0.1 |
| 91 | 0.0825 |
| 92 | 0.105 |
| 39 | 0.1075 |
| 93 | 0.1125 |

TABLE 6

| AON# (SEQ ID NO.) | Average Potency |
|---|---|
| 32 | 0 |
| 65 | 0 |
| 66 | 0 |
| 67 | 0.004115 |
| 68 | 0.070159667 |
| 33 | 0.067908333 |
| 69 | 0.114186333 |
| 70 | 0.469509667 |
| 71 | 0.001819333 |
| 72 | 0.006990667 |
| 34 | 0.00922725 |
| 73 | 0.0024875 |
| 74 | 0.050026 |
| 75 | 0.02369 |
| 76 | 0.011342 |
| 35 | 0.002846 |
| 77 | 0.02729325 |
| 78 | 0.267537 |
| 79 | 0.64751925 |
| 80 | 3.04239475 |
| 36 | 2.17471725 |
| 81 | 0.4560635 |
| 82 | 0.99434725 |
| 83 | 0.66341225 |
| 84 | 0.250514 |
| 37 | 0.0202465 |
| 85 | 0.24768775 |
| 86 | 0.3961975 |
| 87 | 0.402364 |
| 88 | 1.476362 |
| 38 | 1.3001025 |
| 89 | 0.1209145 |
| 90 | 0.0232785 |
| 91 | 0.026413 |
| 92 | 0.0027955 |
| 39 | 0.015113 |
| 93 | 0.005231 |

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

What is claimed is:

1. An AON comprising or consisting of the sequence of any one of AON #1-93 which contains at least one modification.

2. The AON of claim 1 wherein all nucleotides are RNA.

3. The AON of claim 1, wherein the modification is a chemical modification of the sugar moiety of all nucleotides in the AON.

4. The AON of claim 3, wherein the chemical modification of the sugar moiety of all nucleotides in the AON is 2′-MOE.

5. The AON of claim 4, wherein the chemical modification of the sugar moiety is 1, 2, 3 or 4 LNAs, and the remaining nucleotides are 2′-MOE.

6. The AON of claim 5, wherein the chemical modification of the sugar moiety of the two 5′ terminal nucleotide positions of the AON and the two 3′ terminal nucleotide positions of the AON are all LNA, and the remaining nucleotides are 2′-MOE.

7. The AON of claim 3, wherein the chemical modification of the sugar moiety of all nucleotides in the AON is 2′-OMe.

8. The AON of claim 3, wherein the chemical modification of the sugar moiety is 1, 2, 3 or 4 LNAs, and the remaining nucleotides are 2′-OMe.

9. The AON of claim 8, wherein the chemical modification of the sugar moiety of the two 5′ terminal nucleotide positions of the AON and the two 3′ terminal nucleotide positions of the AON are all LNA, and the remaining nucleotides are 2′-OMe.

10. The AON of claim 3, wherein the chemical modification of the sugar moiety of one or more nucleotides in the AON is a morpholine (PMO, PPMO, PMO-X), a peptide derivative (PNA), a boron-cluster modified PNA, a pyrrolidine-based oxy-peptide nucleic acid (POPNA), a glycol- or glycerol-based nucleic acid (GNA), a threose-based nucleic acid (TNA), an acyclic threoninol-based nucleic acid (aTNA), a cationic morpholino-based oligomer (PMOPlus), an oligonucleotide with integrated bases and backbones (ONIBs), a pyrrolidine-amide oligonucleotides (POMs); or a derivative thereof.

11. The AON of claim 1, wherein the modification is a chemical modification of the base moiety of 1, 2, 3, 4 or all nucleotides in the AON.

12. The AON of claim 11, wherein all cytosine bases are replaced with 5-methylcytosine.

13. The AON of claim 11, wherein all thymine bases are replaced with uracil.

14. The AON of claim 1, wherein the backbone is a fully phosphorothioate backbone linkage.

15. The AON of claim 1, wherein the AON comprises a hydroxyalkoxy group at the 5′ terminus of the AON, the 3′ terminus of the AON, or at both the 5′ and 3′ ends of the AON.

16. The AON of claim 15, wherein the hydroxyalkoxy group comprises or consists of an ethylene glycol monomer, ethylene glycol oligomer or ethylene glycol polymer (also known as polyethylene glycol, PEG).

17. The AON of claim 15, wherein the hydroxyalkoxy group is a TEG or a HEG.

18. The AON of claim 1, comprising or consisting of the sequence of AON #33, 34, 35, 36, 37, 38 or 39.

19. The AON of claim 1, comprising or consisting of the sequence of AON #33, 38 or 39.

20. The AON of claim 1, wherein one or two nucleotides are omitted from the 5′ terminus of the AON, or where one or two nucleotides are omitted from the 3′ terminus of the AON, or where one nucleotide is omitted from the 5′ terminus of the AON and one nucleotide is omitted from the 3′ terminus of the AON.

21. The AON of claim 1 that is 16 to 30 nucleotides in length.

22. The AON of claim 1 that is 16, 17, 18, 19 or 20 nucleotides in length.

23. The AON of claim 1 that is 18 nucleotides in length.

24. The AON of claim 1, that is fully 2′-MOE RNA modified, wherein all cytosines are replaced with 5-methylcytosine, and wherein the backbone is a fully phosphorothioate backbone.

25. The AON of claim 1, wherein all cytosines are replaced with 5-methylcytosine, the two 5′ terminal nucleotides of the AON are LNA, the two 3′ terminal nucleotides of the AON are LNA, the remaining nucleotides are 2′-MOE RNA modified, and wherein the backbone is a fully phosphorothioate backbone.

26. The AON of claim 1, that is fully 2′-OMe RNA modified, wherein all cytosines are replaced with 5-methylcytosine, and wherein the backbone is a fully phosphorothioate backbone.

27. The AON of claim 1, wherein all cytosines are replaced with 5-methylcytosine, the two 5′ terminal nucleotides of the AON are LNA, the two 3′ terminal nucleotides of the AON are LNA, the remaining nucleotides are 2′-OMe RNA modified, and wherein the backbone is a fully phosphorothioate backbone.

28. A pharmaceutical composition, comprising the AON of claim 1 and a pharmaceutically acceptable carrier.

29. A method of treating a subject having DMD, comprising administering to the subject the AON of claim 1.

30. A method of delaying the onset of DMD in a subject, comprising administering to the subject the AON of claim 1.

31. A method of inducing skipping of exon 51 of human dystrophin pre-mRNA, comprising contacting human dystrophin pre-mRNA with the AON of claim 1.


SEQUENCE LISTING (96 sequences)

SEQ ID NO: 1 - RNA, 18 nt, synthetic construct
  source 1..18: mol_type = other RNA
aaagaagaaaaagaaaaa

SEQ ID NO: 2 - RNA, 18 nt, synthetic construct
  source 1..18: mol_type = other RNA
gaaaaaagaagaaaaaga

SEQ ID NO: 3 - RNA, 18 nt, synthetic construct
  source 1..18: mol_type = other RNA
aaaaggaaaaaagaagaa

SEQ ID NO: 4 - RNA, 18 nt, synthetic construct
  source 1..18: mol_type = other RNA
ttgcaaaaaggaaaaaag

SEQ ID NO: 5 - RNA, 18 nt, synthetic construct
  source 1..18: mol_type = other RNA
ggtttttgcaaaaaggaa

SEQ ID NO: 6 - RNA, 18 nt, synthetic construct
  source 1..18: mol_type = other RNA
ttttgggtttttgcaaaa

SEQ ID NO: 7 - RNA, 18 nt, synthetic construct
  source 1..18: mol_type = other RNA
aaatattttgggtttttg

SEQ ID NO: 8 - RNA, 18 nt, synthetic construct
  source 1..18: mol_type = other RNA
agctaaaatattttgggt

SEQ ID NO: 9 - RNA, 18 nt, synthetic construct
  source 1..18: mol_type = other RNA
gtaggagctaaaatattt

SEQ ID NO: 10 - RNA, 18 nt, synthetic construct
  source 1..18: mol_type = other RNA
tctgagtaggagctaaaa

SEQ ID NO: 11 - RNA, 18 nt, synthetic construct
  source 1..18: mol_type = other RNA
aacagtctgagtaggagc

SEQ ID NO: 12 - RNA, 18 nt, synthetic construct
  source 1..18: mol_type = other RNA
agagtaacagtctgagta

SEQ ID NO: 13 - RNA, 18 nt, synthetic construct
  source 1..18: mol_type = other RNA
tcaccagagtaacagtct

SEQ ID NO: 14 - RNA, 18 nt, synthetic construct
  source 1..18: mol_type = other RNA
ttgtgtcaccagagtaac

SEQ ID NO: 15 - RNA, 18 nt, synthetic construct
  source 1..18: mol_type = other RNA
acaggttgtgtcaccaga

SEQ ID NO: 16 - RNA, 18 nt, synthetic construct
  source 1..18: mol_type = other RNA
taaccacaggttgtgtca

SEQ ID NO: 17 - RNA, 18 nt, synthetic construct
  source 1..18: mol_type = other RNA
cttagtaaccacaggttg

SEQ ID NO: 18 - RNA, 18 nt, synthetic construct
  source 1..18: mol_type = other RNA
gtttccttagtaaccaca

SEQ ID NO: 19 - RNA, 18 nt, synthetic construct
  source 1..18: mol_type = other RNA
tggcagtttccttagtaa

SEQ ID NO: 20 - RNA, 18 nt, synthetic construct
  source 1..18: mol_type = other RNA
ggagatggcagtttcctt

SEQ ID NO: 21 - RNA, 18 nt, synthetic construct
  source 1..18: mol_type = other RNA
agtttggagatggcagtt

SEQ ID NO: 22 - RNA, 18 nt, synthetic construct
  source 1..18: mol_type = other RNA
tttctagtttggagatgg

SEQ ID NO: 23 - RNA, 18 nt, synthetic construct
  source 1..18: mol_type = other RNA
tggcatttctagtttgga

SEQ ID NO: 24 - RNA, 18 nt, synthetic construct
  source 1..18: mol_type = other RNA
gaagatggcatttctagt

SEQ ID NO: 25 - RNA, 18 nt, synthetic construct
  source 1..18: mol_type = other RNA
tcaaggaagatggcattt

SEQ ID NO: 26 - RNA, 18 nt, synthetic construct
  source 1..18: mol_type = other RNA
caacatcaaggaagatgg

SEQ ID NO: 27 - RNA, 18 nt, synthetic construct
  source 1..18: mol_type = other RNA
acctccaacatcaaggaa

SEQ ID NO: 28 - RNA, 18 nt, synthetic construct
  source 1..18: mol_type = other RNA
caggtacctccaacatca

SEQ ID NO: 29 - RNA, 18 nt, synthetic construct
  source 1..18: mol_type = other RNA
cagagcaggtacctccaa

SEQ ID NO: 30 - RNA, 18 nt, synthetic construct
  source 1..18: mol_type = other RNA
tctgccagagcaggtacc

SEQ ID NO: 31 - RNA, 18 nt, synthetic construct
  source 1..18: mol_type = other RNA
tgaaatctgccagagcag

SEQ ID NO: 32 - RNA, 18 nt, synthetic construct
  source 1..18: mol_type = other RNA
ccggttgaaatctgccag

SEQ ID NO: 33 - RNA, 18 nt, synthetic construct
  source 1..18: mol_type = other RNA
caagcccggttgaaatct

SEQ ID NO: 34 - RNA, 18 nt, synthetic construct
  source 1..18: mol_type = other RNA
ctgtccaagcccggttga

SEQ ID NO: 35 - RNA, 18 nt, synthetic construct
  source 1..18: mol_type = other RNA
aagttctgtccaagcccg

SEQ ID NO: 36 - RNA, 18 nt, synthetic construct
  source 1..18: mol_type = other RNA
tcggtaagttctgtccaa

SEQ ID NO: 37 - RNA, 18 nt, synthetic construct
  source 1..18: mol_type = other RNA
gccagtcggtaagttctg

SEQ ID NO: 38 - RNA, 18 nt, synthetic construct
  source 1..18: mol_type = other RNA
agaaagccagtcggtaag

SEQ ID NO: 39 - RNA, 18 nt, synthetic construct
  source 1..18: mol_type = other RNA
agcagagaaagccagtcg

SEQ ID NO: 40 - RNA, 18 nt, synthetic construct
  source 1..18: mol_type = other RNA
gatcaagcagagaaagcc

SEQ ID NO: 41 - RNA, 18 nt, synthetic construct
  source 1..18: mol_type = other RNA
aacttgatcaagcagaga

SEQ ID NO: 42 - RNA, 18 nt, synthetic construct
  source 1..18: mol_type = other RNA
tttataacttgatcaagc

SEQ ID NO: 43 - RNA, 18 nt, synthetic construct
  source 1..18: mol_type = other RNA
gtgattttataacttgat

SEQ ID NO: 44 - RNA, 18 nt, synthetic construct
  source 1..18: mol_type = other RNA
cctctgtgattttataac

SEQ ID NO: 45 - RNA, 18 nt, synthetic construct
  source 1..18: mol_type = other RNA
atcaccctctgtgatttt

SEQ ID NO: 46 - RNA, 18 nt, synthetic construct
  source 1..18: mol_type = other RNA
ccaccatcaccctctgtg

SEQ ID NO: 47 - RNA, 18 nt, synthetic construct
  source 1..18: mol_type = other RNA
gtcacccaccatcaccct

SEQ ID NO: 48 - RNA, 18 nt, synthetic construct
  source 1..18: mol_type = other RNA
tcaaggtcacccaccatc

SEQ ID NO: 49 - RNA, 18 nt, synthetic construct
  source 1..18: mol_type = other RNA
tatcctcaaggtcaccca

SEQ ID NO: 50 - RNA, 18 nt, synthetic construct
  source 1..18: mol_type = other RNA
gttgatatcctcaaggtc

SEQ ID NO: 51 - RNA, 18 nt, synthetic construct
  source 1..18: mol_type = other RNA
atctcgttgatatcctca

SEQ ID NO: 52 - RNA, 18 nt, synthetic construct
  source 1..18: mol_type = other RNA
tgatcatctcgttgatat

SEQ ID NO: 53 - RNA, 18 nt, synthetic construct
  source 1..18: mol_type = other RNA
cttgatgatcatctcgtt

SEQ ID NO: 54 - RNA, 18 nt, synthetic construct
  source 1..18: mol_type = other RNA
ttctgcttgatgatcatc

SEQ ID NO: 55 - RNA, 18 nt, synthetic construct
  source 1..18: mol_type = other RNA
ataccttctgcttgatga

SEQ ID NO: 56 - RNA, 18 nt, synthetic construct
  source 1..18: mol_type = other RNA
ttctcataccttctgctt

SEQ ID NO: 57 - RNA, 18 nt, synthetic construct
  source 1..18: mol_type = other RNA
attttttctcataccttc

SEQ ID NO: 58 - RNA, 18 nt, synthetic construct
  source 1..18: mol_type = other RNA
ttatcattttttctcata

SEQ ID NO: 59 - RNA, 18 nt, synthetic construct
  source 1..18: mol_type = other RNA
aacttttatcattttttc

SEQ ID NO: 60 - RNA, 18 nt, synthetic construct
  source 1..18: mol_type = other RNA
ctgccaacttttatcatt

SEQ ID NO: 61 - RNA, 18 nt, synthetic construct
  source 1..18: mol_type = other RNA
aacttctgccaactttta

SEQ ID NO: 62 - RNA, 18 nt, synthetic construct
  source 1..18: mol_type = other RNA
agaaaaacttctgccaac

SEQ ID NO: 63 - RNA, 18 nt, synthetic construct
  source 1..18: mol_type = other RNA
tttaaagaaaaacttctg

SEQ ID NO: 64 - RNA, 18 nt, synthetic construct
  source 1..18: mol_type = other RNA
ttcattttaaagaaaaac

SEQ ID NO: 65 - RNA, 18 nt, synthetic construct
  source 1..18: mol_type = other RNA
cccggttgaaatctgcca

SEQ ID NO: 66 - RNA, 18 nt, synthetic construct
  source 1..18: mol_type = other RNA
gcccggttgaaatctgcc

SEQ ID NO: 67 - RNA, 18 nt, synthetic construct
  source 1..18: mol_type = other RNA
agcccggttgaaatctgc

SEQ ID NO: 68 - RNA, 18 nt, synthetic construct
  source 1..18: mol_type = other RNA
aagcccggttgaaatctg

SEQ ID NO: 69 - RNA, 18 nt, synthetic construct
  source 1..18: mol_type = other RNA
ccaagcccggttgaaatc

SEQ ID NO: 70 - RNA, 18 nt, synthetic construct
  source 1..18: mol_type = other RNA
tccaagcccggttgaaat

SEQ ID NO: 71 - RNA, 18 nt, synthetic construct
  source 1..18: mol_type = other RNA
gtccaagcccggttgaaa

SEQ ID NO: 72 - RNA, 18 nt, synthetic construct
  source 1..18: mol_type = other RNA
tgtccaagcccggttgaa

SEQ ID NO: 73 - RNA, 18 nt, synthetic construct
  source 1..18: mol_type = other RNA
tctgtccaagcccggttg

SEQ ID NO: 74 - RNA, 18 nt, synthetic construct
  source 1..18: mol_type = other RNA
ttctgtccaagcccggtt

SEQ ID NO: 75 - RNA, 18 nt, synthetic construct
  source 1..18: mol_type = other RNA
gttctgtccaagcccggt

SEQ ID NO: 76 - RNA, 18 nt, synthetic construct
  source 1..18: mol_type = other RNA
agttctgtccaagcccgg

SEQ ID NO: 77 - RNA, 18 nt, synthetic construct
  source 1..18: mol_type = other RNA
taagttctgtccaagccc

SEQ ID NO: 78 - RNA, 18 nt, synthetic construct
  source 1..18: mol_type = other RNA
gtaagttctgtccaagcc

SEQ ID NO: 79 - RNA, 18 nt, synthetic construct
  source 1..18: mol_type = other RNA
ggtaagttctgtccaagc

SEQ ID NO: 80 - RNA, 18 nt, synthetic construct
  source 1..18: mol_type = other RNA
cggtaagttctgtccaag

SEQ ID NO: 81 - RNA, 18 nt, synthetic construct
  source 1..18: mol_type = other RNA
gtcggtaagttctgtcca

SEQ ID NO: 82 - RNA, 18 nt, synthetic construct
  source 1..18: mol_type = other RNA
agtcggtaagttctgtcc

SEQ ID NO: 83 - RNA, 18 nt, synthetic construct
  source 1..18: mol_type = other RNA
cagtcggtaagttctgtc

SEQ ID NO: 84 - RNA, 18 nt, synthetic construct
  source 1..18: mol_type = other RNA
ccagtcggtaagttctgt

SEQ ID NO: 85 - RNA, 18 nt, synthetic construct
  source 1..18: mol_type = other RNA
agccagtcggtaagttct

SEQ ID NO: 86 - RNA, 18 nt, synthetic construct
  source 1..18: mol_type = other RNA
aagccagtcggtaagttc

SEQ ID NO: 87 - RNA, 18 nt, synthetic construct
  source 1..18: mol_type = other RNA
aaagccagtcggtaagtt

SEQ ID NO: 88 - RNA, 18 nt, synthetic construct
  source 1..18: mol_type = other RNA
gaaagccagtcggtaagt

SEQ ID NO: 89 - RNA, 18 nt, synthetic construct
  source 1..18: mol_type = other RNA
gagaaagccagtcggtaa

SEQ ID NO: 90 - RNA, 18 nt, synthetic construct
  source 1..18: mol_type = other RNA
agagaaagccagtcggta

SEQ ID NO: 91 - RNA, 18 nt, synthetic construct
  source 1..18: mol_type = other RNA
cagagaaagccagtcggt

SEQ ID NO: 92 - RNA, 18 nt, synthetic construct
  source 1..18: mol_type = other RNA
gcagagaaagccagtcgg

SEQ ID NO: 93 - RNA, 18 nt, synthetic construct
  source 1..18: mol_type = other RNA
aagcagagaaagccagtc

SEQ ID NO: 94 - DNA, 19 bp, synthetic construct
  source 1..19: mol_type = other DNA
aaagcagcctgacctagct

SEQ ID NO: 95 - DNA, 24 bp, synthetic construct
  source 1..24: mol_type = other DNA
accactattggagcctttgaaaga

SEQ ID NO: 96 - DNA, 26 bp, synthetic construct
  source 1..26: mol_type = other DNA
cttgtacttcatcccactgattctga